(19) United States
(12) Patent Application Publication
(10) Pub. No.: US 2024/0165788 A1
(43) Pub. Date: May 23, 2024

(54) GRIPPING TOOL

(71) Applicant: Sky Fabrication Inc., Cumberland, RI (US)

(72) Inventor: Danielle Lipsky, Cumberland, RI (US)

(21) Appl. No.: 18/509,669

(22) Filed: Nov. 15, 2023

Related U.S. Application Data
(60) Provisional application No. 63/384,102, filed on Nov. 17, 2022.

(51) Int. Cl. B25J 1/04; A61F 4/00

(52) U.S. Cl. CPC B25J 1/04; A61F 4/00

(57) ABSTRACT

A gripping tool including a first instrument grip with a first connecting node on a first instrument grip planar side and a first instrument channel through the first instrument grip, the first instrument channel parallel to the first instrument grip planar side; a second instrument grip with a second connecting node on a second instrument grip planar side, and a second instrument channel on a second instrument grip spherical side; and a wedge including a first port for receiving the first connecting node and a second port for receiving the second connecting node.

### TECHNICAL FIELD

The subject matter of this invention relates to a gripping tool.

### BACKGROUND

For various reasons a person may have difficulty gripping, grasping, or holding an object with their fingers. For example, limits to strength, dexterity, or other motor or neuro functions in the fingers may also limit the ability to hold writing instruments, make-up tools, eating utensils, and other similar objects.

Similarly, a person without use of their hands may have difficulty gripping, grasping, or holding an object with their toes. Whether fingers or toes, steadying an instrument of any sort between the digits may pose challenges depending upon the instrument, the capabilities of other digits, and the capabilities of other hands or feet.

### SUMMARY

Aspects of the disclosure describe a gripping tool, including a first instrument grip, wherein the first instrument grip includes a first connecting node on a first instrument grip planar side, wherein the first instrument grip includes a first instrument channel through the first instrument grip, and wherein the first instrument channel is parallel to the first instrument grip planar side; a second instrument grip, wherein the second instrument grip includes a second connecting node on a second instrument grip planar side, and wherein the second instrument grip includes a second instrument channel on a second instrument grip spherical side; and a wedge, wherein the wedge includes a first port adapted for receiving the first connecting node and a second port adapted for receiving the second connecting node.

A second aspect discloses a gripping tool, including a first instrument grip, wherein the first instrument grip is torical, wherein the first instrument grip includes a first connecting node on a first instrument grip planar side, wherein the first instrument grip includes a first instrument channel through the first instrument grip, and wherein the first instrument channel is parallel to the first instrument grip planar side; a second instrument grip, wherein the second instrument grip is hemispherical, wherein the second instrument grip includes a second connecting node on a second instrument grip planar side, and wherein the second instrument grip includes a second instrument channel on a second instrument grip spherical side; and a wedge, wherein the wedge includes a first port adapted for receiving the first connecting node and a second port adapted for receiving the second connecting node.

A third aspect discloses a gripping tool wedge system, including a first connecting node; a second connecting node; and a wedge, wherein the wedge is cylindrical, wherein the wedge includes a first port adapted for receiving the first connecting node and the second port adapted for receiving the second connecting node, and wherein the first port and the second port are at opposite ends of the cylinder.

### BRIEF DESCRIPTION OF THE DRAWINGS

These and other features of this invention will be more readily understood from the following detailed description of the various aspects of the invention taken in conjunction with the accompanying figures in which:

FIG. 1 shows a side view of a gripping tool according to embodiments.

FIG. 2 shows a side view of a gripping tool according to embodiments.

FIG. 3 shows a perspective of a view gripping tool according to embodiments.

FIG. 4 shows a side view of wedge for a gripping tool according to embodiments.

FIG. 5 shows a perspective view of wedge for a gripping tool according to embodiments.

FIG. 6 shows a gripping tool in a hand according to embodiments.

FIG. 7 shows a side view of a second instrument grip for a gripping tool according to embodiments.

FIG. 8 shows a bottom view of second instrument grip for a gripping tool according to embodiments.

FIG. 9 shows a top view of second instrument grip for a gripping tool according to embodiments.

FIG. 10 shows a side view of first instrument grip and wedge for a gripping tool according to embodiments.

FIG. 11 shows a perspective and sectional view of a first instrument grip and a wedge for a gripping tool according to embodiments.

FIG. 12 shows a side view of first instrument grip and wedge for a gripping tool according to embodiments.

FIG. 13 shows a perspective and sectional view of a first instrument grip and a wedge for a gripping tool according to embodiments.

FIG. 14 shows a side view of a third instrument grip for a gripping tool according to embodiments.

FIG. 15 shows a top view of a third instrument grip for a gripping tool according to embodiments.

FIG. 16 shows side view of third instrument grip for gripping tool according to embodiments.

FIG. 17 shows bottom view of third instrument grip for gripping tool according to embodiments.

FIG. 18 shows perspective view of third instrument grip for gripping tool according to embodiments.

FIG. 19 shows a side view of gripping tool according to embodiments.

FIG. 20 shows a side view of gripping tool according to embodiments.

FIG. 21 shows a perspective view of gripping tool according to embodiments.

FIG. 22 shows a gripping tool in a hand according to embodiments.

FIG. 23 shows a side view of a gripping tool according to embodiments.

FIG. 24 shows a side view of a gripping tool according to embodiments.

FIG. 25 shows a perspective view of a gripping tool according to embodiments.

FIG. 26 shows perspective views of a gripping tool wedge system according to embodiments.

FIG. 27 shows side sectional views of a gripping tool wedge system according to embodiments.

Any of the images are not necessarily to scale. The images are merely schematic representations and are not intended to portray specific parameters of the invention. The images are intended to depict only typical embodiments of the invention, and therefore should not be considered as limiting the scope of the invention.

### DETAILED DESCRIPTION

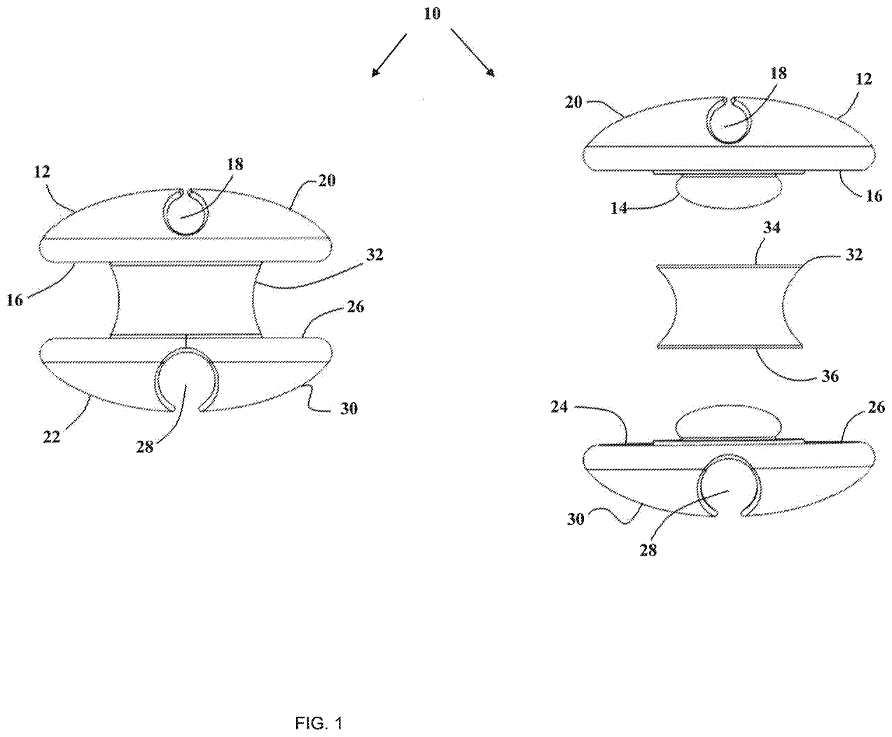

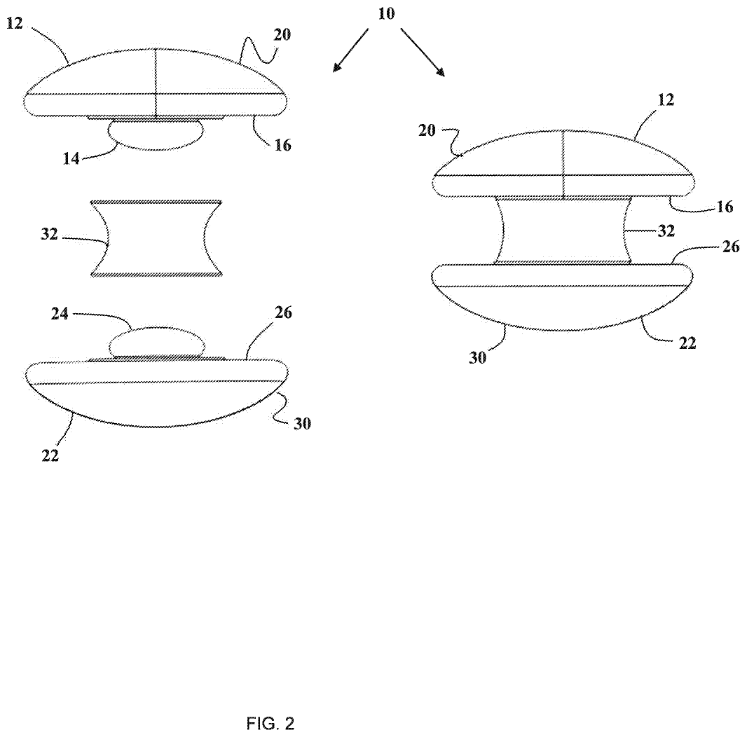

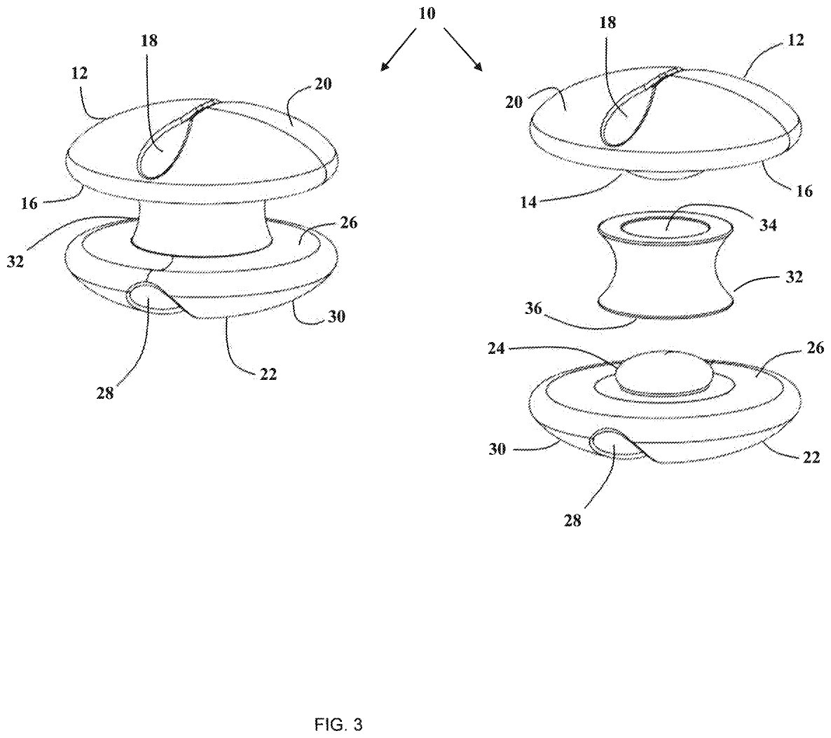

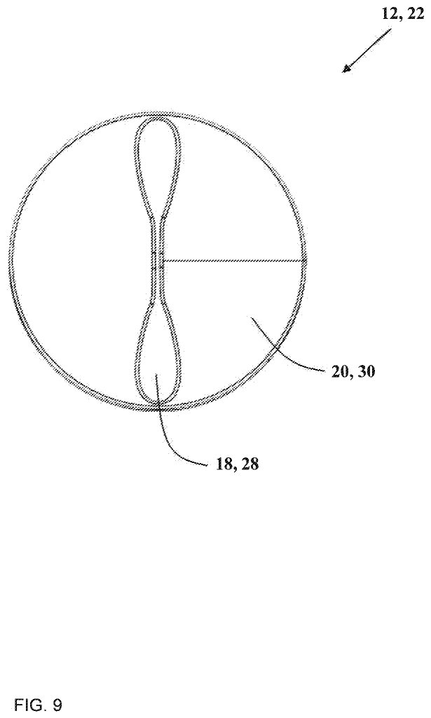

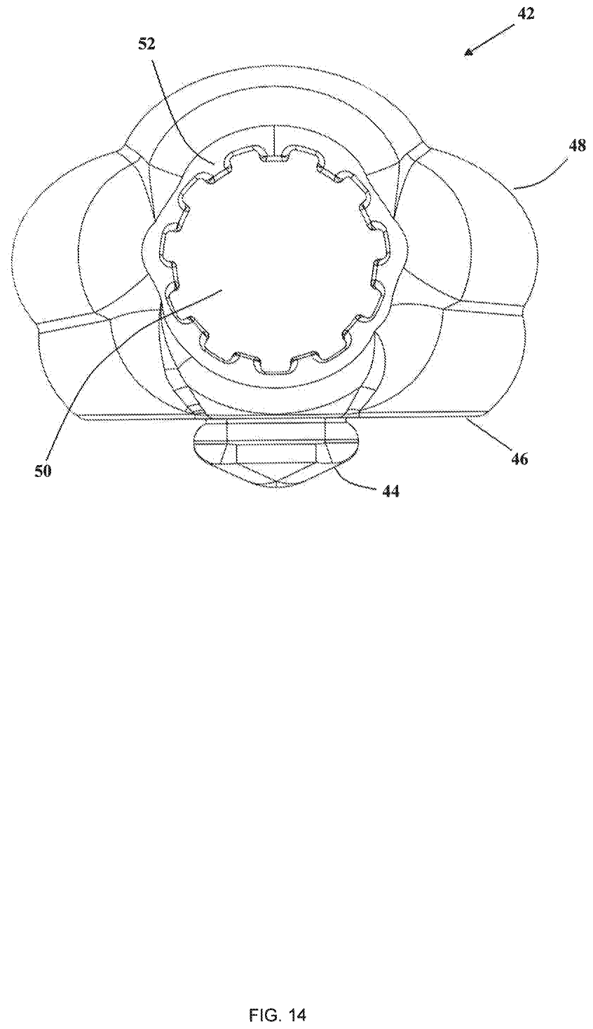

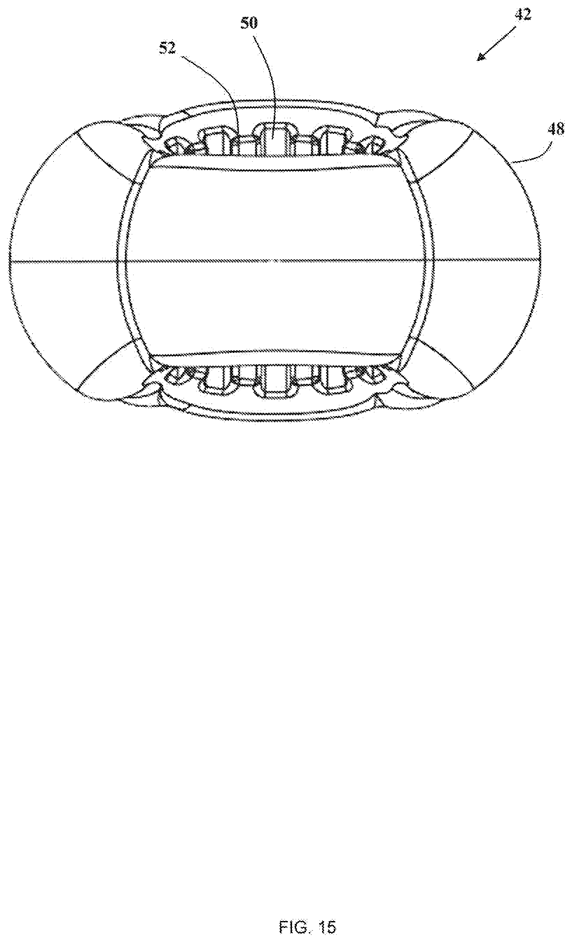

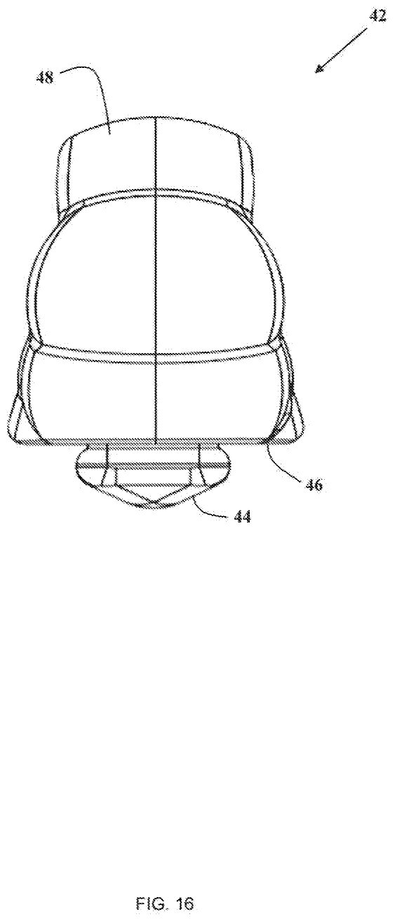

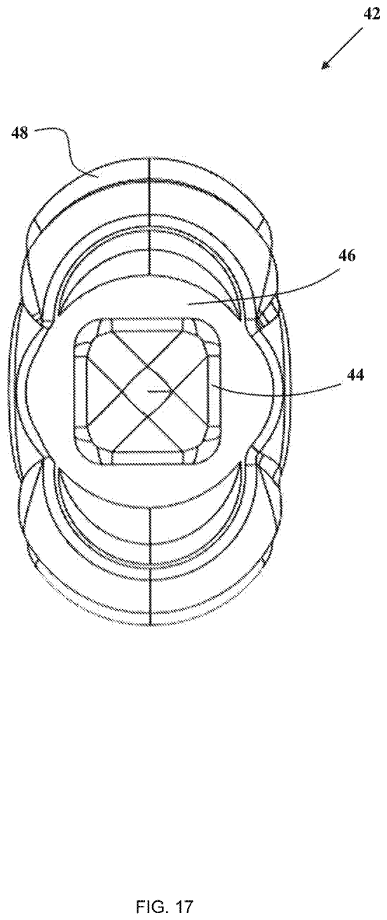

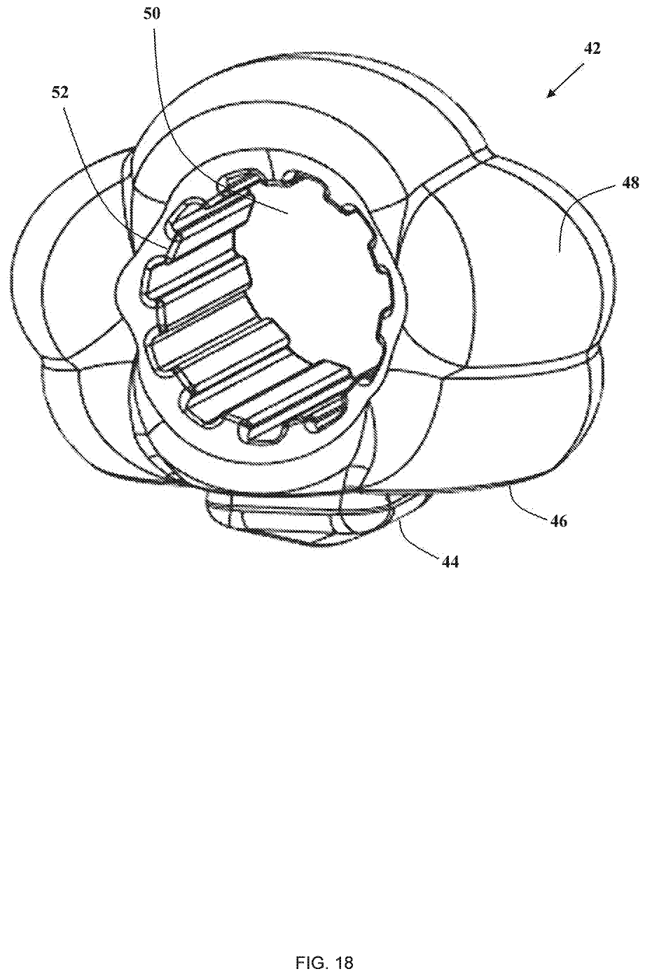

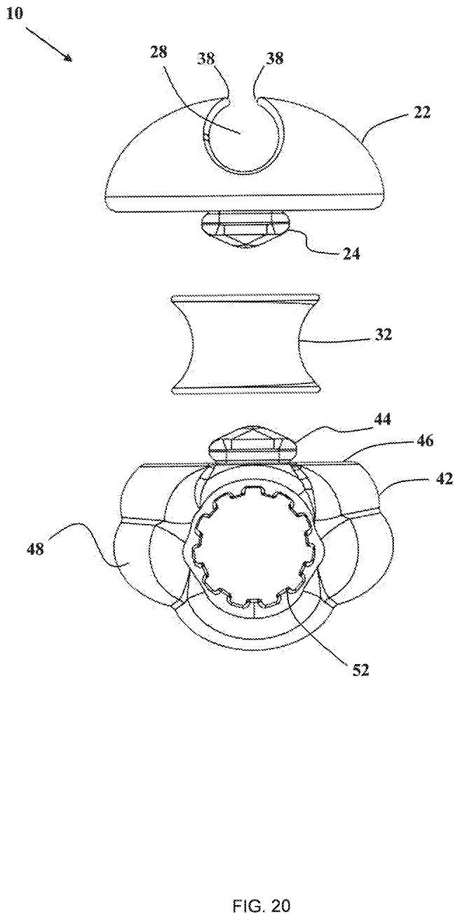

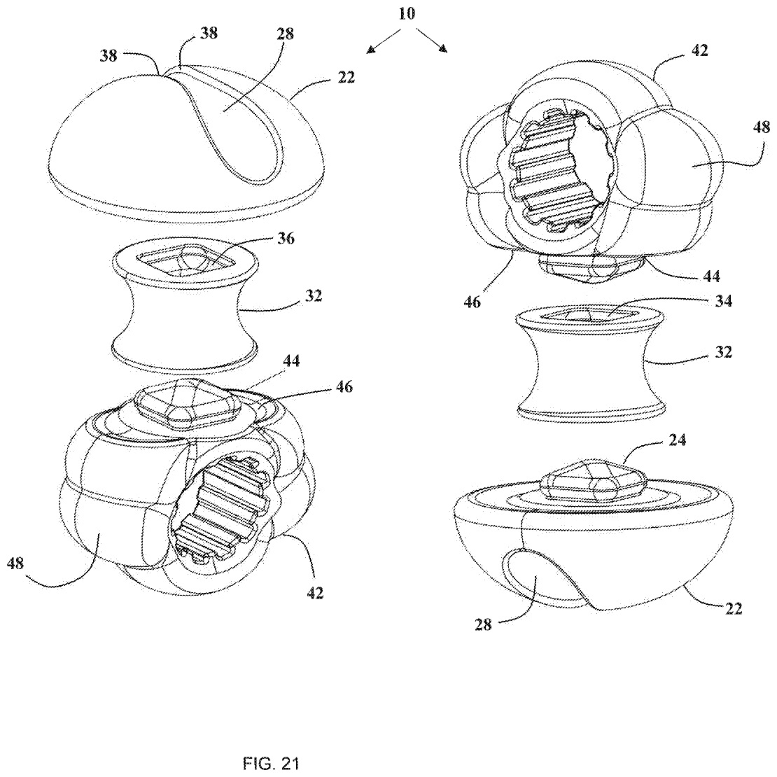

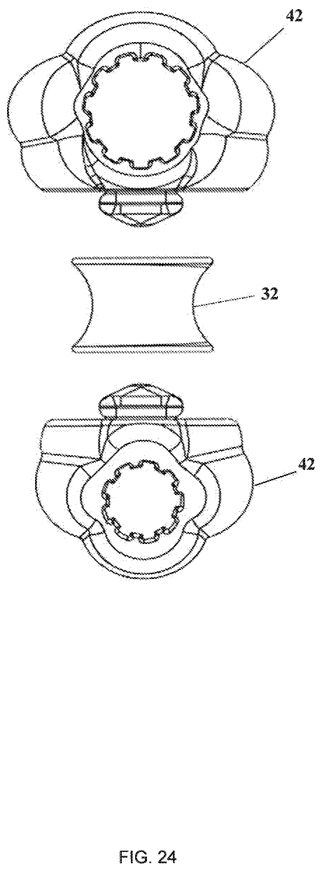

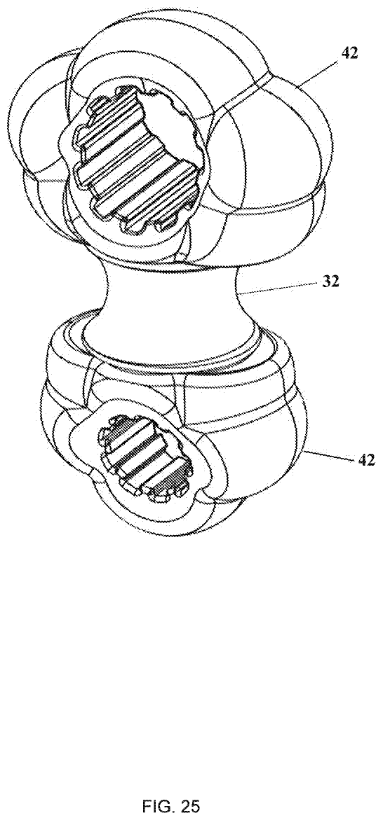

Referring now to the figures, FIGS. 1-25 show various embodiments of a gripping tool 10.

Referring to FIGS. 1-3, one embodiment of the gripping tool 10 may include a first instrument grip 12. The first instrument grip 12 may include a first connecting node 14 on a first planar side 16. The first instrument grip 12 may include a first instrument channel 18 through the first instrument grip 12. The first instrument channel 18 is parallel to the first planar side 16 on a first spherical side 20. The gripping tool 10 may further include a second instrument grip 22. The second instrument grip 22 may include a second connecting node 24 on a second planar side 26. The second instrument grip 22 may include a second instrument channel 28 on a second spherical side 30. The gripping tool 10 may further include a wedge 32. The wedge 32 may include a first port 34 adapted for receiving the first connecting node 14 and a second port 36 adapted for receiving the second connecting node 24.

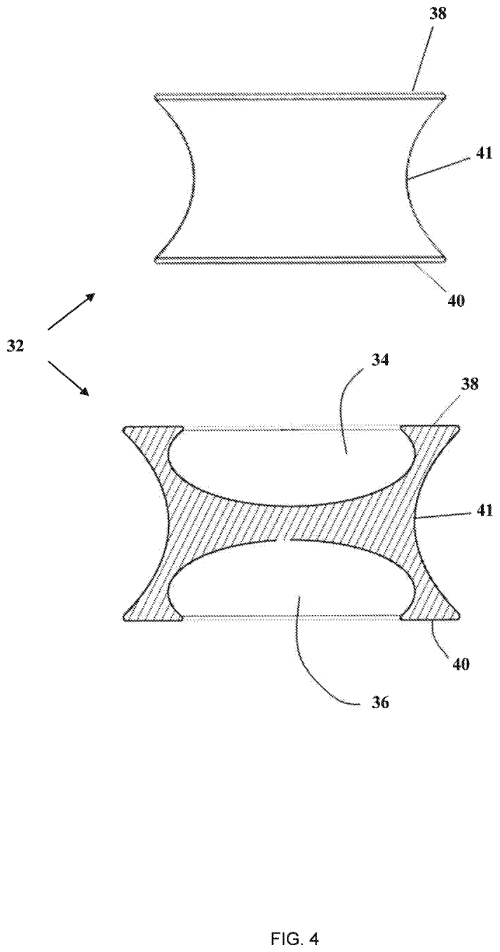

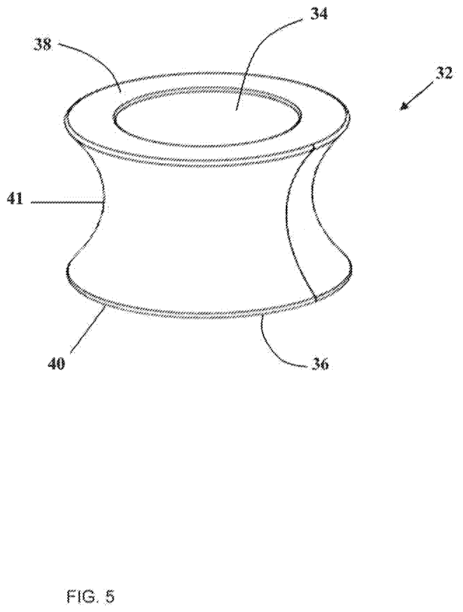

Referring to FIGS. 4-5, wedge 32 may be cylindrical. First port 34 and the second port 36 may be at opposing ends 38, 40 of the wedge 32 with a wedge side 41 connecting the opposing ends 38, 40. The wedge side 41 may curve inward.

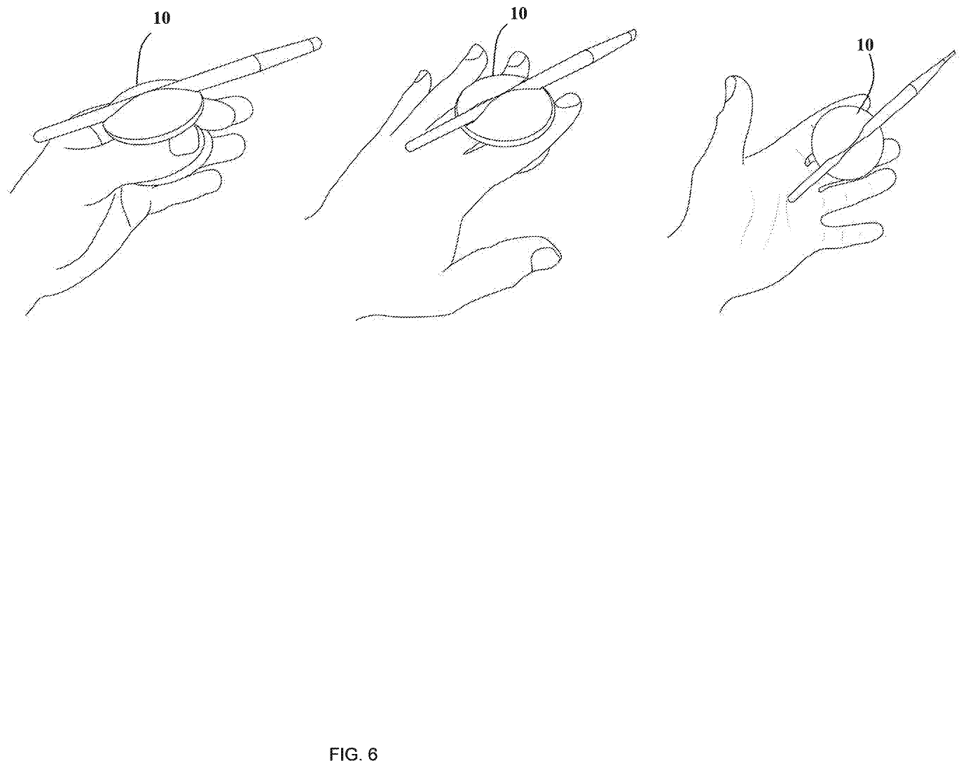

FIG. 6 shows one embodiment of gripping tool 10 in a hand and held between two fingers. This is an example of how gripping tool 10 may be used and not intended to limit other uses of gripping tool 10.

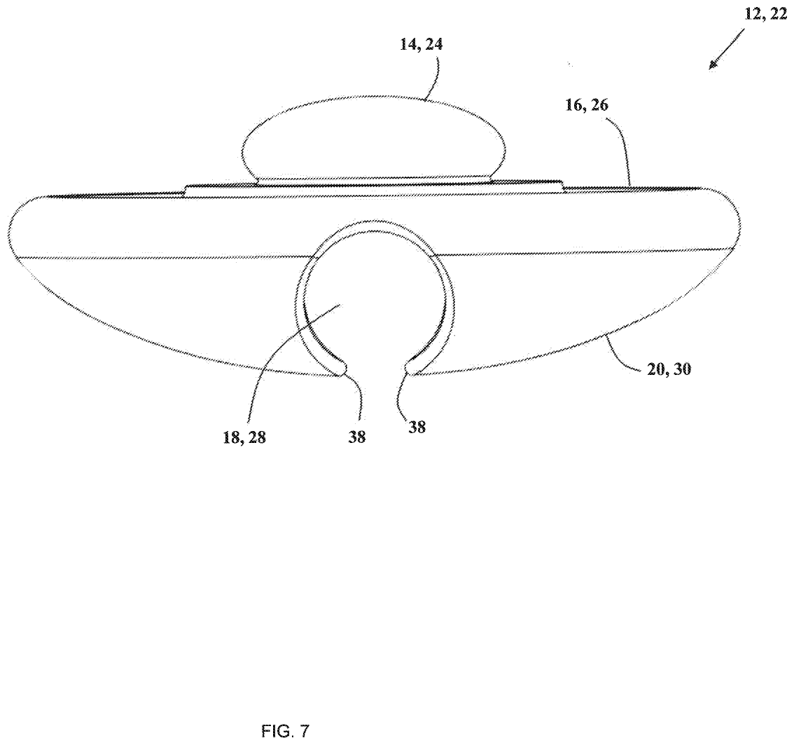

Referring to FIGS. 7-9, first instrument grip 12 and second instrument grip 22 may be hemispherical. First instrument grip 12 and second instrument grip are similarly structured with the comparative size of the first instrument channel 18 and second instrument channel 28 being different. First instrument channel 18 and second instrument channel 28 may form semi-tubular shapes. Semi-tubular shapes may include pliable terminal points 38 adapted for gripping. Pliable terminal points 38 form arcs at the top of the first spherical side 20 and second spherical side 30.

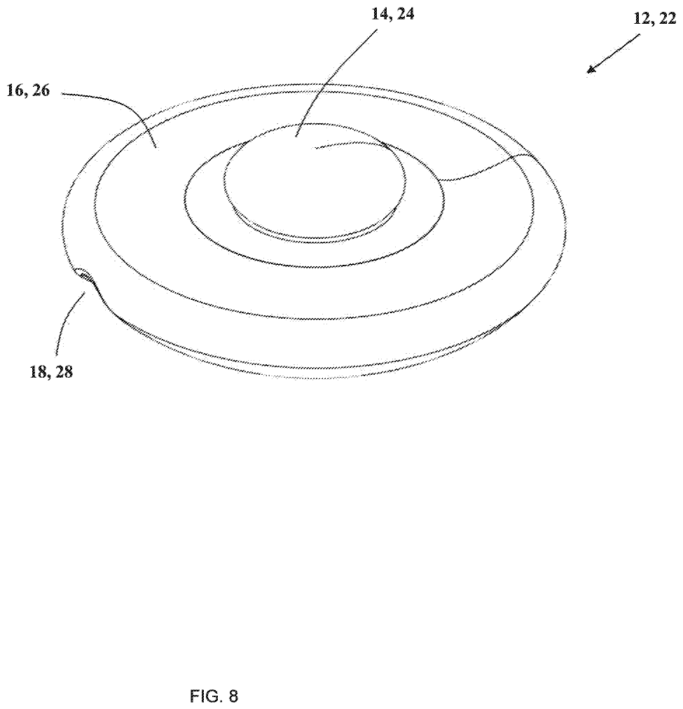

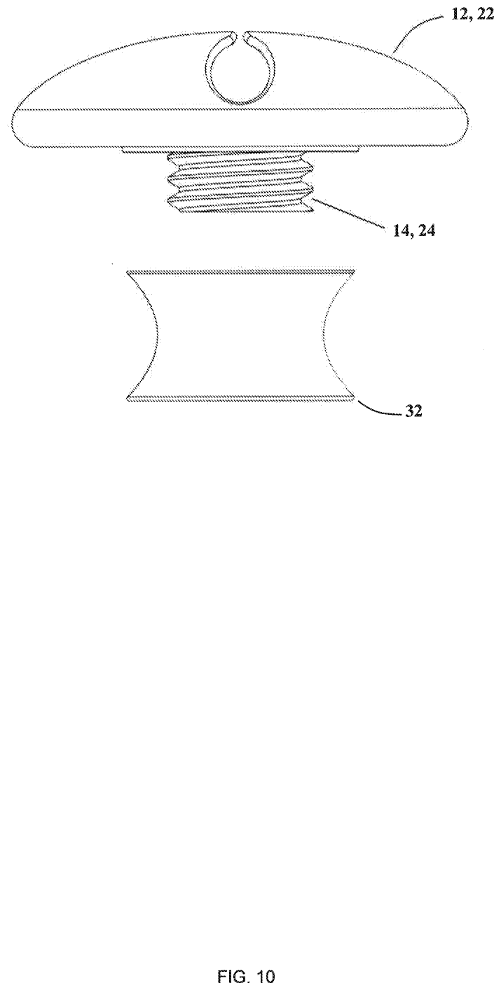

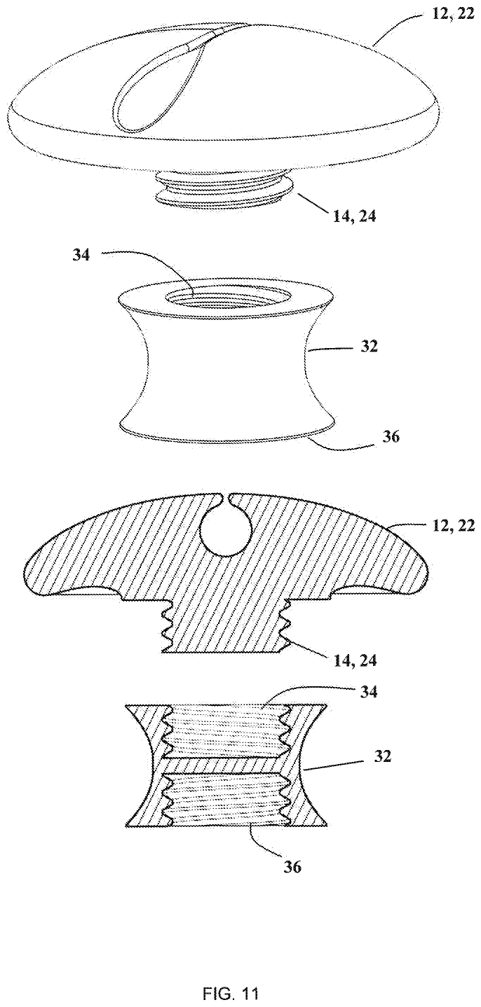

Referring to FIGS. 7-8, first connecting node 14 and second connecting node 24 may be bulbous and the first port 34 and the second port 36 may be similarly shaped bulbous cavities to receive and connect the connecting nodes 14, 24. Referring to FIGS. 10-11, first connecting node 14 and second connecting node 24 may be threaded and the first port 34 and the second port 36 may be similarly threaded to receive and connect the connecting nodes 14, 24.

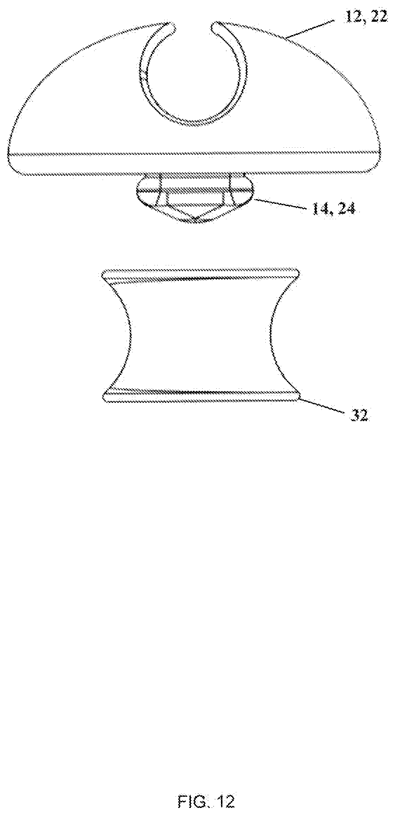

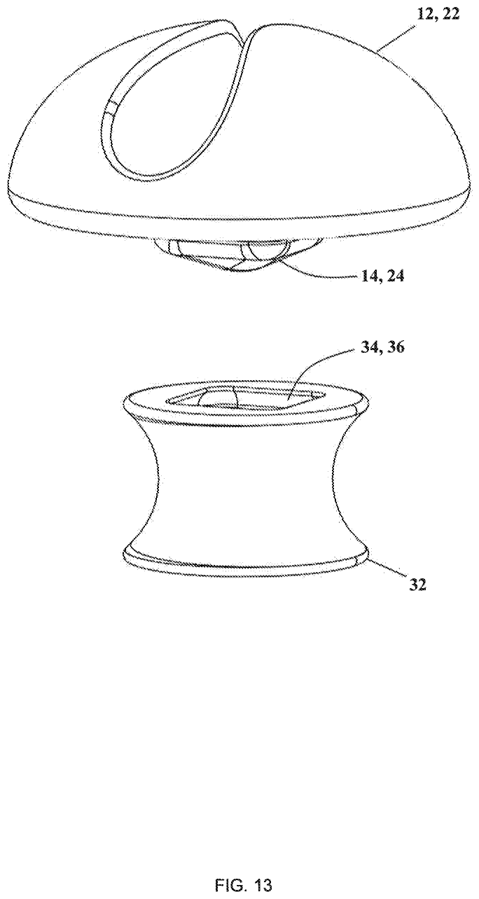

Referring to FIGS. 12-13, first connecting node 14 and second connecting node 24 may be cubic and the first port 34 and the second port 36 may be similarly shaped cubic cavities to receive and connect the connecting nodes 14, 24. A person skilled in the art will readily recognize that connecting nodes 14, 24 and ports 34, 36 may be shaped to provide any number of connections.

First instrument grip 12, second instrument grip 22, and wedge 32 may comprise rubber or silicone or any other similarly pliable material for both holding the gripping tool 10 and for gripping an instrument. Connecting nodes 14, 24, and wedge 32 may be comprised of magnetic materials to facilitate connection.

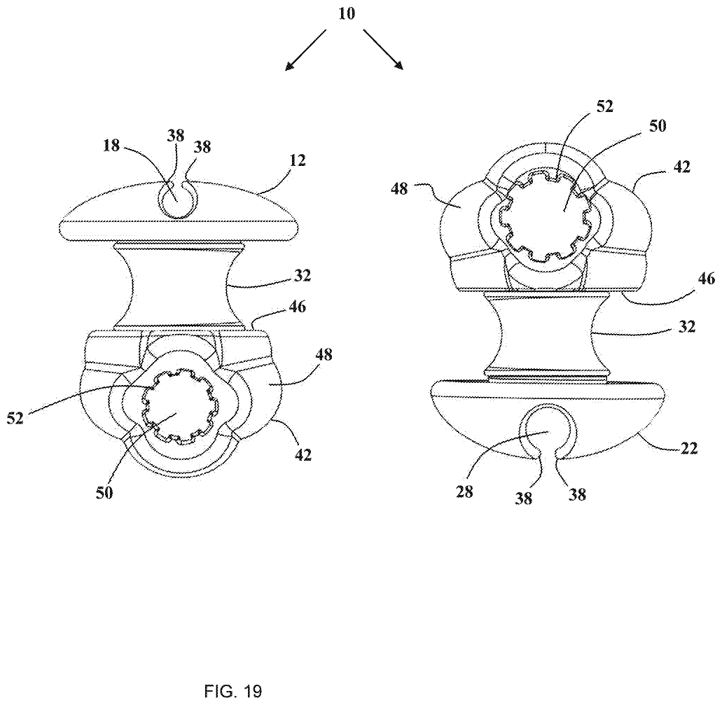

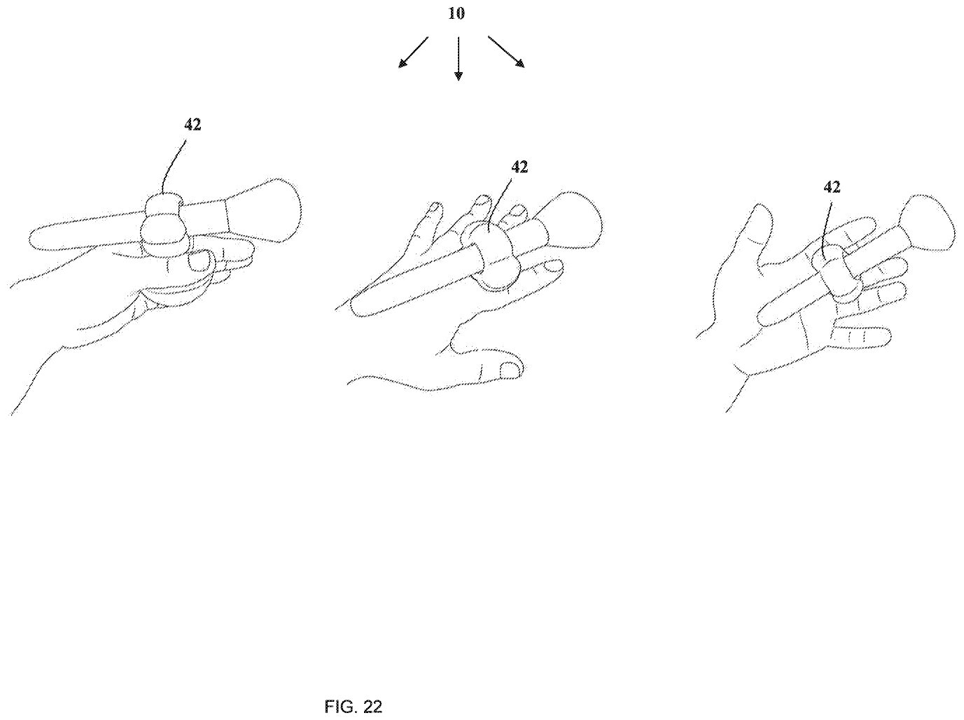

Referring to 14-21, one embodiment of the gripping tool 10 may include a third instrument grip 42. The third instrument grip 42 may be torical. The third instrument grip 42 may include a third connecting node 44 on a third instrument grip planar side 46. The third instrument grip 42 includes a third instrument grip outer surface 48. The third instrument grip outer surface 48 may include a plurality of bulges. The third instrument grip 42 may include a third instrument channel 50 through the third instrument grip 42. The third instrument channel 50 may be parallel to the third instrument grip planar side 46. The gripping tool 10 may further include the second instrument grip 22 (see, FIGS. 19-21). The second instrument grip 22 may be hemispherical. The second instrument grip 22 may include a second connecting node 24 on a second instrument grip planar side 26. The second instrument grip 22 may include a second instrument channel 28 on a second instrument grip spherical side 30. The gripping tool may further include the wedge 32. The wedge 32 may include the first port 34 adapted for receiving the first connecting node 14 and the second port 36 adapted for receiving the second connecting node 24. Wedge 32 may be cylindrical. First port 34 and the second port 36 may be at opposing ends 38, 40 of the wedge 32 with a wedge side 41 connecting the opposing ends 38, 40. The wedge side 41 may curve inward. FIG. 22 shows one embodiment of gripping tool 10 in a hand held between two fingers. This is an example of how gripping tool 10 may be used and not intended to limit other uses of gripping tool 10.

Second instrument channel 28 may form a semi-tubular shape. The semi-tubular shape may include pliable terminal points 38 adapted for gripping. The pliable terminal points 38 may form arcs at the top of the spherical side.

Third instrument channel 48 may form an enclosed tubular shape. Enclosed tubular shape may include pliable teeth 52 circumferentially arranged on the interior of the enclosed tubular shape adapted for gripping.

Third connecting node 44 and second connecting node 24 may be bulbous and the first port 34 and the second port 36 may be similarly shaped bulbous cavities to receive and connect the connecting nodes 44, 24. Third connecting node 44 and second connecting node 24 may be threaded and the first port 34 and the second port 36 may be similarly threaded to receive and connect the connecting nodes 44, 24. Third connecting node 44 and second connecting node may be cubic and the first port 34 and the second port 36 may be similarly shaped cubic cavities to receive and connect the connecting nodes 44, 24. A person skilled in the art will readily recognize that connecting nodes 44, 24 and ports 34, 36 may be shaped to provide any number of connections.

Third instrument grip 42, second instrument grip 22, connecting nodes 44, 14, 24, and wedge 32 may comprise rubber or silicone or any other similarly pliable material for both holding the gripping tool 10 and for gripping an instrument. Connecting nodes 24, 34, 44 and wedge 32 may be comprised of magnetic materials to facilitate connection.

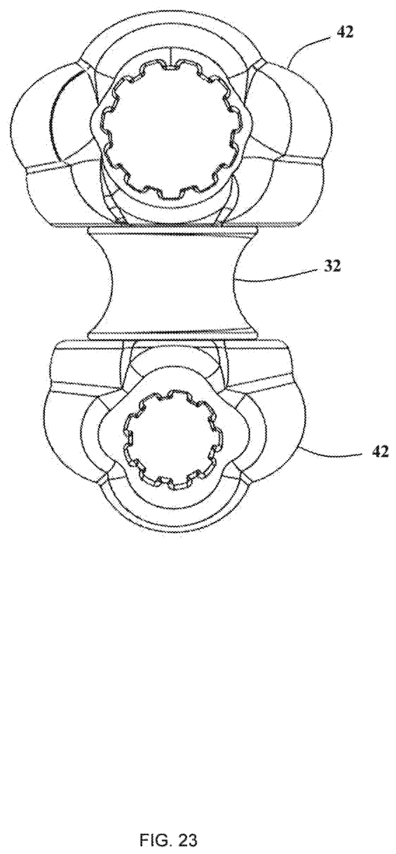

Referring to FIGS. 23-25, a pair of third instrument grips 42 may be connected to the wedge 32, one on each end of wedge 32. A person skilled in the art will readily recognize that first instrument grip 12, second instrument grip 22, and third instrument grip 42 may be connected to wedge 32 in multiple combinations.

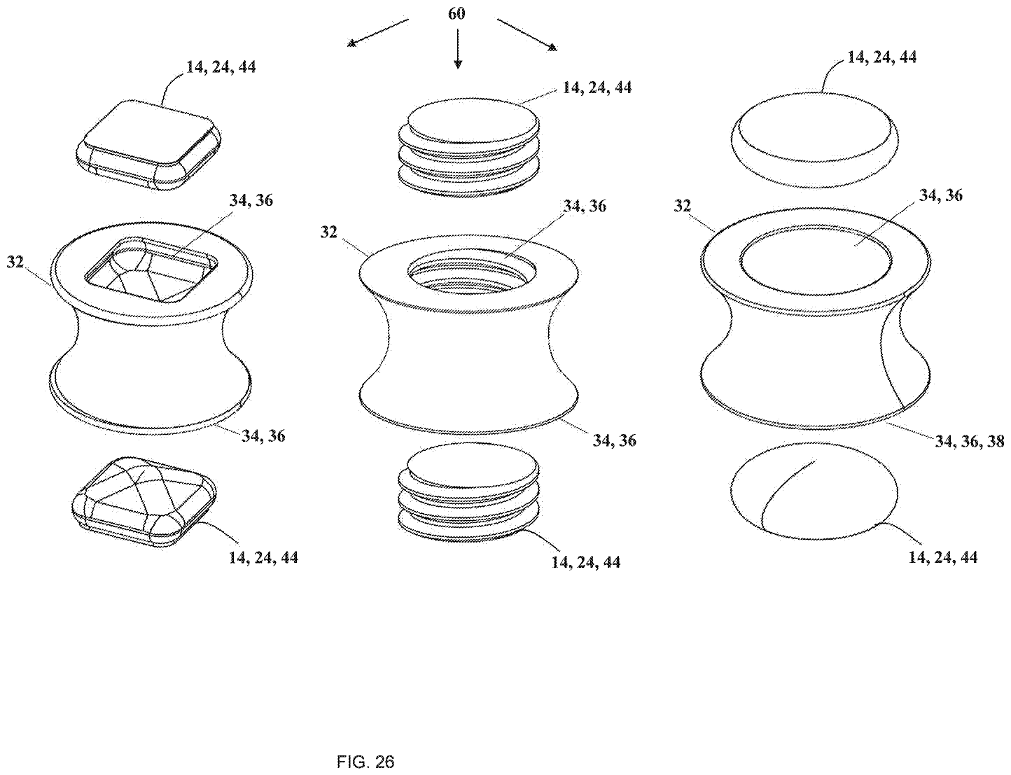

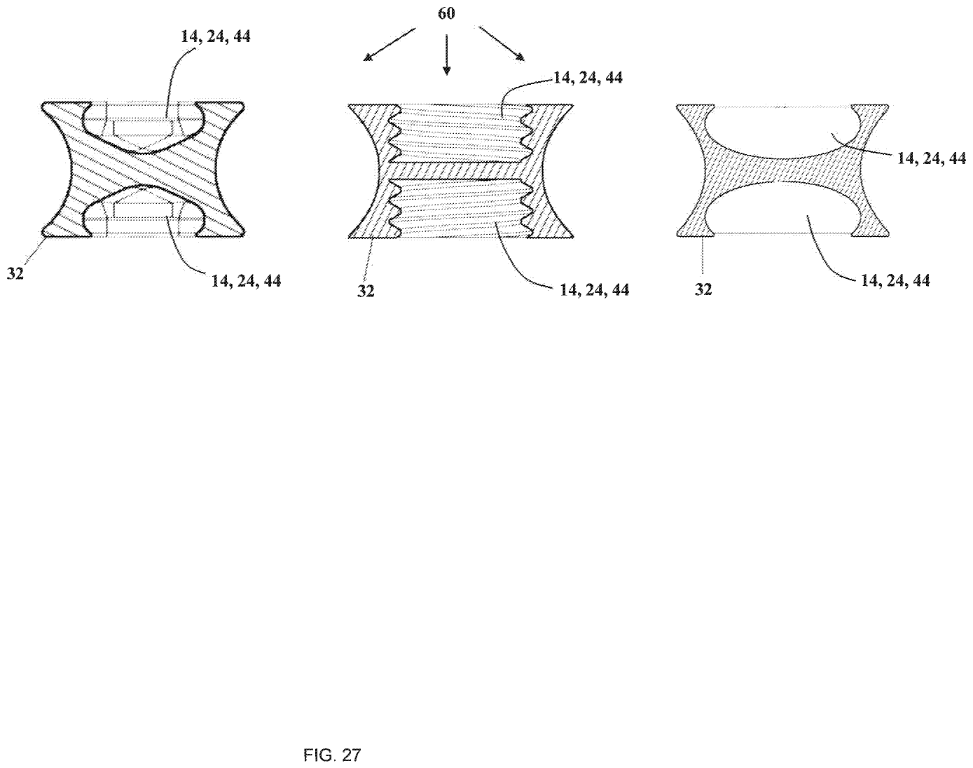

Referring now to the figures, FIGS. 26 and 27 show various embodiments of a gripping tool wedge system 60. Connecting nodes 14, 24, 44, ports 34, 36 and wedge 32 may comprise gripping tool wedge system 60. Gripping tool wedge system, may include a first connecting node 14; a second connecting node 24; third connecting node 44; and a wedge 32, wherein the wedge 32 is cylindrical, wherein the wedge 32 includes a first port 34 adapted for receiving the first connecting node 14, second connecting node 24, or third connecting node 44 and the second port 36 adapted for receiving for receiving the first connecting node 14, second connecting node 24, or third connecting node 44, and wherein the first port 34 and the second port 36 are at opposite ends 38, 40 of the wedge 32. As discussed herein, the connecting nodes 24, 34, 44 and ports 34, 36 may be bulbous, threaded, or cubic as shown in FIGS. 26 and 27. A person skilled in the art will readily recognize that other shapes for the connecting nodes 24, 14, 44, and ports 34, 36 may be utilized.

One embodiment includes a method of deploying a gripping tool 10 including first instrument grip 12, second instrument grip 22, and wedge 32 as described herein and connecting the first connecting node 14 to first port 34 and second connecting node to second port 36 of wedge 32.

One embodiment includes a gripping tool system including first instrument grip 12, second instrument grip 22, and wedge 32 as described herein that may be connected to deploy gripping tool 10 by first connecting node 14 to first port 34 and connecting the second connecting node 24 to second port 36.

One embodiment includes a method of deploying a gripping tool 10 including third instrument grip 42, second instrument grip 22, and wedge 32 as described herein and connecting the third connecting node 44 to first port 34 and second connecting node to second port 36 of wedge 32.

One embodiment includes a gripping tool system including third instrument grip 42, second instrument grip 22, and wedge 32 as described herein that may be connected to deploy gripping tool 10 by third connecting node 44 to first port 34 and connecting the second connecting node 24 to second port 36.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A gripping tool, comprising:
a first instrument grip, wherein the first instrument grip includes a first connecting node on a first instrument grip planar side, wherein the first instrument grip includes a first instrument channel through the first instrument grip, and wherein the first instrument channel is parallel to the first instrument grip planar side;
a second instrument grip, wherein the second instrument grip includes a second connecting node on a second instrument grip planar side, and wherein the second instrument grip includes a second instrument channel on a second instrument grip spherical side; and
a wedge, wherein the wedge includes a first port adapted for receiving the first connecting node and a second port adapted for receiving the second connecting node.

2. The gripping tool of claim 1, wherein the first connecting node and the second connecting node are cubic.

3. The gripping tool of claim 1, wherein the first connecting node and the second connecting node are bulbous.

4. The gripping tool of claim 1, wherein the first connecting node and the second connecting node are threaded.

5. The gripping tool of claim 1, wherein the first instrument grip and the second instrument grip are hemispherical.

6. The gripping tool of claim 1, wherein the first instrument channel and the second instrument channel form semi-tubular shapes.

7. The gripping tool of claim 6, wherein the semi-tubular shapes include pliable terminal points adapted for gripping.

8. The gripping tool of claim 7, wherein the pliable terminal points form arcs at the top of the spherical side.

9. The gripping tool of claim 1, wherein the wedge is a cylinder, and wherein the first port and the second port are at opposite ends of the cylinder.

10. The gripping tool of claim 1, wherein the sides of the cylinder curve inward.

11. A gripping tool, comprising:
a first instrument grip, wherein the first instrument grip is torical, wherein the first instrument grip includes a first connecting node on a first instrument grip planar side, wherein the first instrument grip includes a first instrument channel through the first instrument grip, and wherein the first instrument channel is parallel to the first instrument grip planar side;
a second instrument grip, wherein the second instrument grip is hemispherical, wherein the second instrument grip includes a second connecting node on a second instrument grip planar side, and wherein the second instrument grip includes a second instrument channel on a second instrument grip spherical side; and
a wedge, wherein the wedge includes a first port adapted for receiving the first connecting node and the second port adapted for receiving the second connecting node.

12. The gripping tool of claim 11, wherein the first connecting node and the second connecting node are cubic.

13. The gripping tool of claim 11, wherein the first connecting node and the second connecting node are bulbous.

14. The gripping tool of claim 11, wherein the first connecting node and the second connecting node are threaded.

15. The gripping tool of claim 11, wherein the second instrument channel forms a semi-tubular shape, wherein the semi-tubular shape includes pliable terminal points adapted for gripping, and wherein the pliable terminal points form arcs at the top of the spherical side.

16. The gripping tool of claim 15, wherein the pliable terminal points form arcs at the top of the spherical side.

17. The gripping tool of claim 11, wherein the first instrument channel forms an enclosed tubular shape.

18. The gripping tool of claim 16, wherein the enclosed tubular shape includes pliable teeth circumferentially arranged on the interior of the enclosed tubular shape adapted for gripping.

19. The gripping tool of claim 11, wherein the wedge is a cylinder, and wherein the first port and the second port are at opposite ends of the cylinder.

20. A gripping tool wedge system, comprising:
a first connecting node;
a second connecting node; and
a wedge, wherein the wedge is cylindrical, wherein the wedge includes a first port adapted for receiving the first connecting node and the second port adapted for receiving the second connecting node, and wherein the first port and the second port are at opposite ends of the cylinder.
